(19) United States
(12) Patent Application Publication
(10) Pub. No.: US 2024/0165300 A1
(43) Pub. Date: May 23, 2024

(54) 3D PRINTED SCAFFOLDS FOR USE IN TISSUE REPAIR

(71) Applicant: AEVUMED, INC., Malvern, PA (US)

(72) Inventors: Saif Khalil, Malvern, PA (US); Miles Curtis, Berwyn, PA (US); Robert P. Douglass, Bryn Mawr, PA (US)

(21) Appl. No.: 18/512,727

(22) Filed: Nov. 17, 2023

Related U.S. Application Data
(60) Provisional application No. 63/384,339, filed on Nov. 18, 2022.

(51) Int. Cl. A61L 27/24; A61L 27/26

(52) U.S. Cl. CPC A61L 27/24; A61L 27/26; A61L 2430/10

(57) ABSTRACT

A printable, biocompatible scaffold is described, having a first layer comprising a first material, and a second layer comprising a second material that is different from the first material. The scaffold includes a first region having a first thickness and a second region having a second thickness that is greater than the thickness of the first region.

### CROSS-REFERENCE TO RELATED APPLICATIONS

This application claims priority to U.S. provisional application No. 63/384,339 filed Nov. 18, 2022 incorporated herein by reference in its entirety.

### BACKGROUND OF THE INVENTION

Soft tissue tears are common and many repair methods rely solely on the mechanical attributes of fasteners to repair the soft tissue. However, the fasteners alone are often inadequate for proper healing, as the soft tissue is in a weakened state and penetrating the soft tissue with the fasteners merely introduces additional weak points that are prone to further tearing.

3D printing is an emerging manufacturing technique that offers great precision to control the architecture of implantable scaffolds. Based on computer-aided design (CAD) models, 3D printers can fabricate a biocompatible construct in a layer-by-layer fashion. 3D printing and rapid prototyping processes have been used to create scaffolds with user defined micro-structures and micro-scaled architectures. However, these systems still lack higher sophistication both in the ability to control and define scaffold architecture.

Thus, there is a need in the art for improved scaffolds to utilize with anchoring systems to enhance repair and healing. The present invention meets this need.

### BRIEF DESCRIPTION OF THE DRAWINGS

The following detailed description of preferred embodiments of the invention will be better understood when read in conjunction with the appended drawings. For the purpose of illustrating the invention, there are shown in the drawings embodiments which are presently preferred. It should be understood, however, that the invention is not limited to the precise arrangements and instrumentalities of the embodiments shown in the drawings.

FIG. 1 is an image of an exemplary 3D printing system depositing material in formation of a scaffold layer according to an aspect of the present invention.

FIGS. 2A and 2B are a schematic of exemplary scaffolds having two or more regions within the scaffold.

FIG. 3 is a schematic of an exemplary scaffold and suture anchoring device with a scaffold loaded into the device.

FIG. 4 is an image of a scaffold being positioned onto soft tissue via the device of FIG. 3.

FIG. 5 is an image of the positioned scaffold onto the soft tissue and between the soft tissue and bone.

FIG. 6 is an image of the scaffold being secured to the soft tissue and bone via bone anchors and sutures.

### DETAILED DESCRIPTION

### Definitions

Unless defined otherwise, all technical and scientific terms used herein have the same meaning as commonly understood by one of ordinary skill in the art to which this invention belongs. Any methods and materials similar or equivalent to those described herein can be used in the practice or testing of the present invention.

As used herein, each of the following terms has the meaning associated with it in this section.

The articles “a” and “an” are used herein to refer to one or to more than one (i.e., to at least one) of the grammatical object of the article. By way of example, “an element” means one element or more than one element.

“About” as used herein when referring to a measurable value, for example numerical values and/or ranges, such as an amount, a temporal duration, and the like, is meant to encompass variations of ±20%, ±10%, ±5%, ±1%, or ±0.1% from the specified value, as such variations are appropriate to perform the disclosed methods. For example, “about 40 [units]” may mean within ±25% of 40 (e.g., from 30 to 50), within ±20%, ±15%, ±10%, ±9%, ±8%, ±7%, ±6%, ±5%, ±4%, ±3%, ±2%, ±1%, less than ±1%, or any other value or range of values therein or therebelow. Furthermore, the phrases “less than about [a value]” or “greater than about [a value]” should be understood in view of the definition of the term “about” provided herein.

Throughout this disclosure, various aspects of the invention can be presented in a range format. It should be understood that the description in range format is merely for convenience and brevity and should not be construed as an inflexible limitation on the scope of the invention. Accordingly, the description of a range should be considered to have specifically disclosed all the possible sub-ranges as well as individual numerical values within that range. For example, description of a range such as from 1 to 6 should be considered to have specifically disclosed sub-ranges such as from 1 to 3, from 1 to 4, from 1 to 5, from 2 to 4, from 2 to 6, from 3 to 6 etc., as well as individual numbers within that range, for example, 1, 2, 2.7, 3, 4, 5, 5.3, and 6. This applies regardless of the breadth of the range.

### Detailed Description

The present invention relates in part to biocompatible and implantable scaffolds. In some embodiments, the scaffolds are tailored for tendon repair, including tendon-to-tendon and/or tendon-to-bone applications. In some embodiments, the scaffolds form homogenous or non-homogenous, single layer or multi-layered constructs. The present invention also relates in part to methods for depositing natural and/or synthetic polymers to create any of the biocompatible and implantable scaffolds described herein.

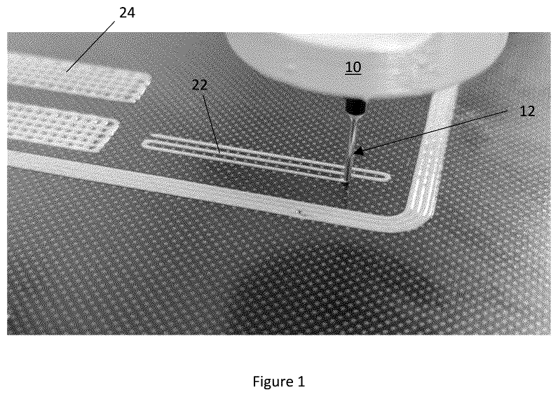

As contemplated herein, the scaffolds may be constructed from one or more layers of deposited material. FIG. 1 shows a 3D printer 10 having a deposition needle 12 with a distal tip opening sized to deposit the desired amount of material in either droplets or as a continuously flowing strand 22 to form one or more scaffold layers 24. In some embodiments, an additional support material is deposited onto or within the spacing of a scaffold layer. In some embodiments, such support materials may effectively fill the spacing between strands, or otherwise fully fill or at least partially fill the porosity of any previously deposited material in the scaffold. In some embodiments, the support material may form part of the final scaffold structure or it may be removed from the final scaffold mechanically or chemically, for example via heating, cooling, drying, dissolving and the like.

In some embodiments, certain individual strands of the scaffold may have an average diameter of less than 2 mm, less than 1.8 mm, less than 1.6 mm, less than 1.4 mm, less than 1.2 mm, less than 1 mm, less than 0.8 mm, less than 0.6 mm, less than 0.4 mm, less than 0.2 mm or less than 0.1 mm. In some embodiments, the average diameter of certain individual strands of the scaffold may have an average diameter of between 0.1 mm and 2 mm. In some embodiments, the strands of the scaffold have about the same average diameter. In some embodiments, the strands of the scaffold have a variable average diameter. For example, in some embodiments, 90% of the strands have an average diameter of less than 2 mm and 50% of the strands have an average diameter of less than 1 mm.

In some embodiments, the spacing between certain adjacent individual strands of the scaffold may have an average distance of less than 2 mm, less than 1.8 mm, less than 1.6 mm, less than 1.4 mm, less than 1.2 mm, less than 1 mm, less than 0.8 mm, less than 0.6 mm, less than 0.4 mm, less than 0.2 mm or less than 0.1 mm. In some embodiments, the average distance between certain adjacent individual strands of the scaffold may have an average distance of between 0.1 mm and 2 mm. In some embodiments, the adjacent strands of the scaffold have about the same average distance apart. In some embodiments, the adjacent strands of the scaffold have a variable average distance apart. For example, in some embodiments, 90% of adjacent strands have an average distance apart of less than 2 mm and 50% of adjacent strands have an average distance apart of less than 1 mm.

As mentioned previously, the scaffolds of the present invention may include one or more layers of deposited material. In some embodiments, the scaffold includes multiple regions, where a first region has more layers than a second region. In some embodiments, the strand orientation between layers may be between 0° and 90°, between 0° and 80°, between 0° and 70°, between 0° and 60°, between 0° and 50°, between 0° and 40°, between 0° and 30°, between 0° and 20°, between 0° and 10°, or between 0° and 5°. In some embodiments, the strand orientation between layers may be about 5°, about 10°, about 15°, about 20°, about 25°, about 30°, about 35°, about 40°, about 45°, about 50°, about 55°, about 60°, about 65°, about 70°, about 75°, about 80° or about 85°. In some embodiments, the strand orientation between layers may be substantially parallel. In some embodiments, the strand orientation between layers may be substantially perpendicular. In some embodiments, the strands may be oriented in a grid-like structure layer by layer. In some embodiments, each layer may have the same or a different strand diameter and spacing to an adjacent layer. In some embodiments, one or more layers may or may not include a support material. The support material for each layer may entirely fill or at least partially fill voids within the strands of one or more layers of the scaffold.

In various embodiments, scaffolds of the present invention can have any desired shape, including but not limited to square, rectangular, polygonal, circular, ovoid, and irregularly shaped sheets. The scaffolds of the present invention may be rigid, flexible, clastic, or combinations thereof. For example, the scaffold may include multiple regions, where a first region is rigid or semi-rigid, and a second region is flexible, semi-flexible and/or elastic. In some embodiments, the scaffolds may include one or more hinging regions, such that the scaffold is configured to bend or fold.

In some embodiments, the scaffold has an overall porosity of between 50% and 99.9%. In some embodiments, the scaffold has an overall porosity of about 50%, greater than 60%, greater than 70%, greater than 80%, greater than 90%, greater than 92%, greater than 94%, greater than 96%, greater than 98%, greater than 99%, greater than 99.2%, greater than 99.4%, greater than 99.6%, greater than 99.8% or about 99.9%. In some embodiments, any particular layer of the scaffold has an overall porosity of between 50% and 99.9%. In some embodiments, the particular scaffold layer has an overall porosity of about 50%, greater than 60%, greater than 70%, greater than 80%, greater than 90%, greater than 92%, greater than 94%, greater than 96%, greater than 98%, greater than 99%, greater than 99.2%, greater than 99.4%, greater than 99.6%. greater than 99.8% or about 99.9%. In some embodiments, the porosity of adjacent layers is same or may be different. In some embodiments, the scaffold porosity may be induced or enhanced via lyophilization.

In some embodiments, the scaffold may have a length of between about 1 mm and 50 mm, between about 2 mm and 50 mm, between about 5 mm and 50 mm, between about 1 mm and 40 mm, between about 2 mm and 40 mm, between about 5 mm and 40 mm, between about 10 mm and 30 mm or between about 10 mm and 20 mm. In some embodiments, the scaffold may have a length of less than 50 mm, less than 45 mm, less than 40 mm, less than 35 mm, less than 30 mm, less than 25 mm, less than 20 mm, less than 15 mm, less than 10 mm, less than 5 mm, less than 4 mm, less than 3 mm, less than 2 mm, or less than 1 mm.

In some embodiments, the scaffold may have a width of between about 1 mm and 50 mm, between about 2 mm and 50 mm, between about 5 mm and 50 mm, between about 1 mm and 40 mm, between about 2 mm and 40 mm, between about 5 mm and 40 mm, between about 10 mm and 30 mm or between about 10 mm and 20 mm. In some embodiments, the scaffold may have a width of less than 50 mm, less than 45 mm, less than 40 mm, less than 35 mm, less than 30 mm, less than 25 mm, less than 20 mm, less than 15 mm, less than 10 mm, less than 5 mm, less than 4 mm, less than 3 mm, less than 2 mm, or less than 1 mm.

In some embodiments, the scaffold may have a thickness of between about 0.01 mm and 5 mm, between about 0.02 mm and 5 mm, between about 0.05 mm and 5 mm, between about 0.01 mm and 4 mm, between about 0.02 mm and 4 mm, between about 0.05 mm and 4 mm, between about 0.1 mm and 3 mm or between about 0.1 mm and 2 mm. The thickness of any portion of the scaffold may be based on the material composition of the strands and/or support material, as well as the number of layers forming the scaffold. Each layer of the scaffold has a thickness and is formed by one or more strands and/or support material. In some embodiments, the scaffold may have a thickness of less than 5 mm, less than 4.5 mm, less than 4.0 mm, less than 3.5 mm, less than 3.0 mm, less than 2.5 mm, less than 2.0 mm, less than 1.5 mm, less than 1.0 mm, less than 0.5 mm, less than 0.4 mm, less than 0.3 mm, less than 0.2 mm, less than 0.1 mm, less than 0.09 mm, less than 0.08 mm, less than 0.07 mm, less than 0.06 mm, less than 0.05 mm, less than 0.04 mm, less than 0.03 mm, less than 0.02 mm, or less than 0.01 mm. In some embodiments, the scaffold has a uniform thickness. In some embodiments, the scaffold has variable thickness.

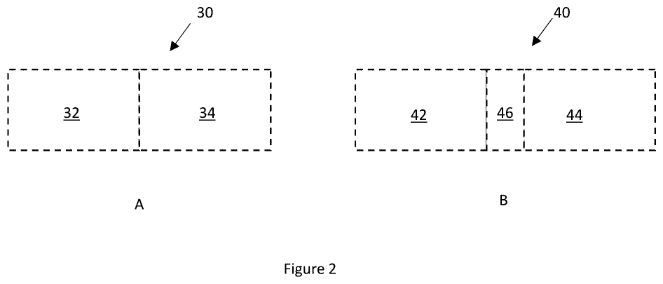

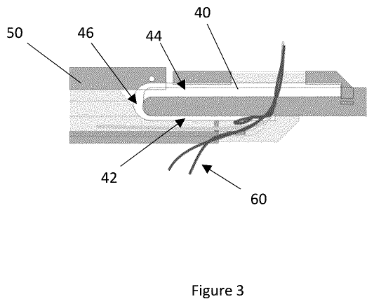

In some embodiments, the scaffold has a first region of a first thickness, and a second region of a second thickness. It should be appreciated that the scaffold may have any number of regions, each having a respective thickness and functional mechanical property (such as rigidity, flexibility, elasticity, material or mechanical strength, etc.). For example, as shown in FIG. 2A, the scaffold 30 may have a first region 32 having a first thickness, and a second region 34 having a second thickness, where the thickness of region 34 is greater than or equal to the thickness of region 32. Such exemplary scaffolds may be suitable for and configured to provide more or less mechanical strength based in part on the thickness of each region when implanted into a subject. Further, by having regions of greater or lesser thickness can promote better fit on top of or between certain tissues when implanted in a subject. In some embodiments, the thickness of the second region is at least 10% the thickness of the first region, at least 15% the thickness of the first region, at least 20% the thickness of the first region, at least 25% the thickness of the first region, at least 30% the thickness of the first region, at least 35% the thickness of the first region, at least 40% the thickness of the first region, at least 45% the thickness of the first region, at least 50% the thickness of the first region, at least 60% the thickness of the first region, at least 70% the thickness of the first region, at least 80% the thickness of the first region, at least 90% the thickness of the first region, at least 100% the thickness of the first region, or at least 200% the thickness of the first region. As shown in FIG. 2B, the scaffold 40 may have a first region 42 having a first thickness, a second region 44 having a second thickness, and a third region 46 having a third thickness, where the thickness of region 44 is greater than or equal to the thickness of region 42, and where the thickness of region 42 is greater than or equal to region 46. Exemplary scaffolds of FIG. 2B are shown positioned within a scaffold and suture anchoring device in FIG. 3, and may be configured to fold at region 46, while regions 42 and 44 are configured to accept and support one or more sutures passing through the scaffold and neighboring or adjacent biological tissues and/or bone anchors.

In some embodiments, the scaffold has first and second regions with different strand orientations. These regions can have the same thickness or different thicknesses. Variation in strand orientation can apply to scaffolds with any number of regions, each having a respective strand orientation that corresponds to at least one of a functional mechanical property (such as rigidity, flexibility, elasticity, material or mechanical strength, etc.), an anatomy of the patient, or the surgeons preferred implantation pattern, e.g. creating more strength where sutures pierce the scaffold, or creating a recessed area on the scaffold for sutures to rest in. For example, the scaffold may have a first region having a first strand orientation, and a second region having a second strand orientation, and the thickness of region could be the same or different than the thickness of region. Such exemplary scaffolds may be suitable for and configured to provide more or less mechanical strength based in part on the strand orientation of each region when implanted into a subject. The different strand orientations may also provide more rigidity or flexibility in certain directions, which depending on the anatomy of the patient and the implantation technique can promote better performing custom implant.

Scaffolds of the present invention can comprise synthetic materials, biological materials, and combinations thereof to enhance biocompatibility and healing. In some embodiments, strands and/or support material of the scaffold may be composed of a single material. In some embodiments, strands and/or support material of the scaffold may be composed of multiple materials. In some embodiments, the strand and/or support material is a synthetic material. In some embodiments, the strand and/or support material is a biological material. In some embodiments, the strand and/or support material is a composite. In some embodiments, the strand and/or support material is a combination of synthetic and biological materials. As contemplated herein, each layer of the scaffold may be formed of strands composed of one or more materials.

Contemplated synthetic strand and/or support materials include but are not limited to: poly(urethanes), poly(siloxanes) or silicones, poly(ethylene), poly(vinyl pyrrolidone), poly(2-hydroxy ethyl methacrylate), poly(N-vinyl pyrrolidone), poly(methyl methacrylate), poly(vinyl alcohol), poly(acrylic acid), polyacrylamide, poly(ethylene-co-vinyl acetate), poly(ethylene glycol), poly(methacrylic acid), polylactic acid (PLA), poly(L-lactic acid) (PLLA), polyglycolic acids (PGA), poly(lactide-co-glycolides) (PLGA), polycaprilactone (PCL), tri-calcium phosphate (TCP), polycaprilactone-tri-calcium phosphate (PCL-TCP), nylons, polyamides, polyanhydrides, poly(ethylene-co-vinyl alcohol) (EVOH), polycaprolactone, poly(vinyl acetate) (PVA), polyvinylhydroxide, poly(ethylene oxide) (PEO), polyorthoesters, any combinations thereof or any other similar synthetic polymers that may be developed that are biologically compatible. Contemplated biological strand and/or support materials such as proteins, enzymes and cells may be included. Other examples include but are not limited to: collagen (e.g. Type I with Type II,

Type I with Type III, Type II with Type III, etc.), fibrin, fibrinogen, thrombin, elastin, laminin, fibronectin, hyaluronic acid, chondroitin 4-sulfate, chondroitin 6-sulfate, dermatan sulfate, gelatin, heparin sulfate, heparin, and keratan sulfate, proteoglycans, polysaccharides (e.g. cellulose and its derivatives), chitin, chitosan, alginic acids, and alginates such as calcium alginate and sodium alginate, and any combinations of biological and/or synthetic materials. In some embodiments, scaffolds of the present invention comprise tissue grafts. In some embodiments, scaffolds of the present invention comprise isotropic materials. In other embodiments, scaffolds of the present invention comprise anisotropic fibers, such that they can be positioned in a direction that aligns anisotropic fibers in a direction of natural or expected anatomic forces to resist tearing and further damage.

In various embodiments, scaffolds of the present invention can be embedded or conjugated with factors that promote healing, including but not limited to growth factors such as epidermal growth factor (EGF), platelet derived growth factor (PDGF), basic fibroblast growth factor (bFGF), transforming growth factor-β (TGF-β), and tissue inhibitors of metalloproteinases (TIMP). Additional factors can include antibiotics, bacteriocides, fungicides, silver-containing agents, analgesics, and nitric oxide releasing compounds. Scaffolds of the present invention may also be seeded with proteins, enzymes, cells, such as fibroblasts, osteoblasts, keratinocytes, epithelial cells, endothelial cells, mesenchymal stem cells, and/or embryonic stem cells.

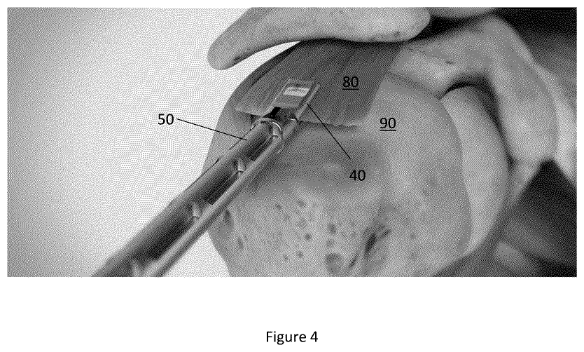

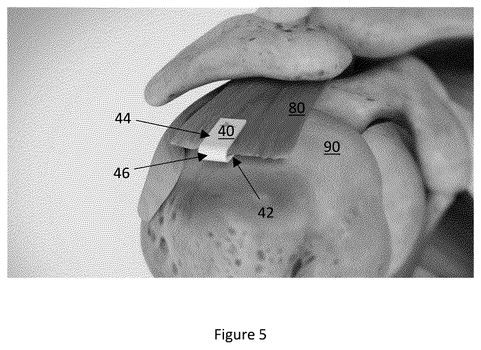

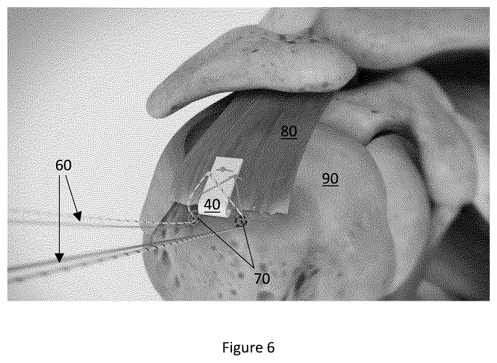

Scaffolds of the present invention can be configured to heal or repair a target site. For example, as shown in FIGS. 4-6, scaffolds of the present invention can be used to wrap around a soft tissue such as a tendon or ligament for secure attachment to a bone surface. In a first step and as shown in FIG. 4, the scaffold and suture anchoring device 50 is loaded with a scaffold 40 in a manner similar to as shown in FIG. 3. Scaffold 40 is positioned about soft tissue 80. As can be seen in FIG. 5, scaffold 40 is uniquely structurally designed such that a thin region 42 of scaffold 40 presents a lower profile when positioned between bone 90 and the bottom surface of soft tissue 80 to minimize lifting of soft tissue 80 from bone 90. Region 46 of scaffold 40 thickens and permits folding around the peripheral edge of soft tissue 80. As shown in FIG. 6, region 44 is thicker and provides enhanced mechanical strength for tying and securing any number of sutures 60 and/or bone anchors 70 needed to properly secure the soft tissue the bone.

Embodiments of the 3D printer may perform electrocompaction on the collagen media, which promotes organization and alignment of the collagen molecules by electrical charge. Advantageously, this increases the bond strength of the collagen membrane during the 3D printing process. In some embodiments, the methods of depositing natural and/or synthetic materials to create the scaffold may comprise aligning the molecules of the materials used. Aligning the molecules may be achieved through electrocompaction, electrospinning, gravity-based alignment, extrusion-based alignment, or any methods known in the art. In some embodiments, aligning the molecules is achieved through electrocompaction. In some embodiments, electrocompaction is performed by the 3D printer. In some embodiments, electrocompaction further comprises generating an electric field between two electrodes across a solution of the scaffold material. As an example, the electric field generates a pH gradient and charges the support material molecules such that they align and compact at an isoelectric point. In some embodiments, the electrodes used may be of any suitable shape depending on the shape of the scaffold. For example, and without limitation, linear, curvilinear, planar, curviplanar, or tubular electrodes may be used. In some embodiments, electrocompaction promotes organization and alignment in the scaffold material, leading to an increase in mechanical strength of the scaffold.

The disclosures of each and every patent, patent application, and publication cited herein are hereby incorporated herein by reference in their entirety. While this invention has been disclosed with reference to specific embodiments, it is apparent that other embodiments and variations of this invention may be devised by others skilled in the art without departing from the true spirit and scope of the invention. The appended claims are intended to be construed to include all such embodiments and equivalent variations.

What is claimed is:

1. A printable, biocompatible scaffold comprising:
a first layer comprising a first material; and
a second layer comprising a second material that is different from the first material;
wherein the scaffold includes a first region having a first thickness and a second region having a second thickness that is greater than the thickness of the first region.

2. The scaffold of claim 1, further comprising a first support material integrated into the first layer.

3. The scaffold of claim 1, further comprising a second support material integrated into the second layer.

4. The scaffold of claim 1, wherein the first material is a synthetic polymer.

5. The scaffold of claim 1, wherein the second material is a synthetic polymer.

6. The scaffold of claim 4 or 5, wherein the synthetic polymer is selected from the group consisting of poly(urethanes), poly(siloxanes) or silicones, poly(ethylene), poly(vinyl pyrrolidone), poly(2-hydroxy ethyl methacrylate), poly(N-vinyl pyrrolidone), poly(methyl methacrylate), poly(vinyl alcohol), poly(acrylic acid), polyacrylamide, poly(ethylene-co-vinyl acetate), poly(ethylene glycol), poly(methacrylic acid), polylactic acid (PLA), poly(L-lactic acid) (PLLA), polyglycolic acids (PGA), poly(lactide-co-glycolides) (PLGA), polycaprilactone (PCL), tri-calcium phosphate (TCP), polycaprilactone-tri-calcium phosphate (PCL-TCP), nylons, polyamides, polyanhydrides, poly(ethylene-co-vinyl alcohol) (EVOH), polycaprolactone, poly(vinyl acetate) (PVA), polyvinylhydroxide, poly(ethylene oxide) (PEO), polyorthoesters, any combinations thereof.

7. The scaffold of claim 1, wherein the first material is a biological material.

8. The scaffold of claim 1, wherein the second material is a biological material.

9. The scaffold of claim 8, wherein the biological material is selected from the group consisting of collagen, fibrin, fibrinogen, thrombin, elastin, laminin, fibronectin, hyaluronic acid, chondroitin 4-sulfate, chondroitin 6-sulfate, dermatan sulfate, gelatin, heparin sulfate, heparin, keratan sulfate, proteoglycans, polysaccharides, chitin, chitosan, alginic acids, and alginates and any combinations thereof.

10. The scaffold of claims 9, wherein the first layer is a synthetic polymer and the second layer is a biological material.

11. The scaffold of claim 8, wherein the biological material is collagen applied via electrocompaction.

12. The scaffold of claim 1, wherein the first or second layer comprises both a synthetic polymer and a biological material.
